(19) United States
(12) Patent Application Publication
(10) Pub. No.: US 2025/0049974 A1
(43) Pub. Date: Feb. 13, 2025

(54) FAR UV-C DISINFECTION LIGHTING, SYSTEMS AND METHODS

(71) Applicant: FAR UV TECHNOLOGIES, INC., Kansas City, MO (US)

(72) Inventors: Patrick J. PIPER, Kansas City, MO (US); Michael L. CLARK, Saint Augustine, FL (US); Christopher A. `WIKLOF, Everett, WA (US)

(21) Appl. No.: 18/787,448

(22) Filed: Jul. 29, 2024

Related U.S. Application Data
(63) Continuation-in-part of application No. 18/626,734, filed on Apr. 4, 2024, now abandoned.
Application No. 18/626,734 is a continuation of application No. PCT/US2022/077822, filed on Oct. 7, 2022.
(63) Continuation-in-part of application No. PCT/US2023/061608, filed on Jan. 30, 2023.
(63) Continuation-in-part of application No. PCT/US2023/065515, filed on Apr. 7, 2023.
(63) Continuation of application No. PCT/US2023/064268, filed on Mar. 13, 2023.
(63) Continuation-in-part of application No. PCT/US2023/064269, filed on Mar. 13, 2023.
(60) Provisional application No. 63/253,162, filed on Oct. 7, 2021.
(60) Provisional application No. 63/267,272, filed on Jan. 28, 2022.
(60) Provisional application No. 63/348,439, filed on Jun. 2, 2022.
(60) Provisional application No. 63/269,257, filed on Mar. 13, 2022.
(60) Provisional application No. 63/269,257, filed on Mar. 13, 2022.
(60) Provisional application No. 63/578,793, filed on Aug. 25, 2023.

(51) Int. Cl. A61L 2/10; A61L 2/24; A61L 2/26; A61L 9/20

(52) U.S. Cl. CPC A61L 2/10; A61L 2/24; A61L 2/26; A61L 9/20; A61L 2202/11; A61L 2202/14; A61L 2202/16; A61L 2202/25; A61L 2209/111; A61L 2209/12; A61L 2209/15

(57) ABSTRACT

Embodiments provide improved devices, systems, and methods of disinfection that use ultraviolet light, specifically, far UV-C light, to kill or deactivate microbes or allergens that are present on various types of surfaces and/or in the air.

### CROSS-REFERENCE TO RELATED APPLICATIONS

The present application is a Continuation-in-Part of co-pending U.S. patent application Ser. No. 18/626,734, filed Apr. 4, 2024 entitled “IMPROVED DISINFECTION LIGHTING SYSTEMS AND METHODS”; which is a Continuation of International Patent Application No. PCT/US2022/077,822 entitled “IMPROVED DISINFECTION LIGHTING SYSTEMS AND METHODS” filed Oct. 7, 2022 (Docket No. 3083-001-04), now expired; which claims priority benefit from U.S. Provisional Patent Application No. 63/253,162, entitled “IMPROVED DISINFECTION LIGHTING SYSTEMS AND METHODS,” filed on Oct. 7, 2021 (Docket No. 3083-001-02), now expired.

The present application is a Continuation-in-Part of co-pending International Patent Application No. PCT/US2023/061,608, entitled “ULTRAVIOLET ILLUMINATOR WITH NETWORK COMMUNICATION,” filed on Jan. 30, 2023 (Docket No. 3082-003-04); which claims priority benefit from U.S. Provisional Patent Application No. 63/267,272, entitled “ULTRAVIOLET ILLUMINATOR WITH NETWORK COMMUNICATION,” filed on Jan. 28, 2022 (Docket No. 3082-003-02), now expired.

The present application is a Continuation-in-Part of co-pending International Patent Application No. PCT/US2023/065,515, entitled “FAR ULTRAVIOLET LAMP AND SYSTEM WITH ILLUMINATION DIFFUSER,” filed on Apr. 7, 2023 (Docket No.: 3083-004-04); which claims priority benefit from U.S. Provisional Patent Application No. 63/348,439, entitled “FAR ULTRAVIOLET LAMP AND SYSTEM WITH ILLUMINATION DIFFUSER,” filed on Jun. 2, 2022 (Docket No. 3083-004-02), now expired.

The present application is a Continuation-in-Part of co-pending International Patent Application No. PCT/US2023/064,268, entitled “EXCIMER ILLUMINATOR WITH REPLACEABLE LAMP,” filed on Mar. 13, 2023 (Docket No, 3083-006-04); which claims priority benefit from U.S. Provisional Patent Application No. 63/269,257, entitled “EXCIMER ILLUMINATOR WITH REPLACEABLE LAMP,” filed on Mar. 13, 2022 (Docket No. 3083-006-02), now expired.

The present application is a Continuation-in-Part of co-pending International Patent Application No. PCT/US2023/064,269, entitled “REPLACEABLE EXCIMER LAMP,” filed on Mar. 13, 2023 (Docket No. 3083-011-04); which claims priority benefit from U.S. Provisional Patent Application No. 63/269,257, entitled “EXCIMER ILLUMINATOR WITH REPLACEABLE LAMP,” filed on Mar. 13, 2022 (Docket No. 3083-006-02), now expired.

The present application claims priority benefit from co-pending U.S. Provisional Patent Application No. 63/578,793, entitled “UVC ILLUMINATOR WITH OZONE MITIGATION,” filed on Aug. 25, 2023 (Docket No. 3083-012-02).

Each of the foregoing applications is incorporated by reference in its entirety.

### BACKGROUND

### Field of the Invention

Embodiments relate to improved disinfection devices and their associated uses and methods that use of a narrow spectrum of ultraviolet light to eliminate biological contaminants in air and on surfaces.

### Description of the Related Art

Implementing, operating, and maintaining a healthy and safe environment is of paramount importance in any facility, as well as areas that are likely to be sources of pathogens. Current disinfection solutions are costly, require time-consuming processes and have limited efficacy. The level and type of infectious agents and other contaminants are also factors contributing to costs and challenges in maintaining safe and healthy environments.

Ultraviolet (UV) light is germicidal and has been used for disinfection since the mid-20th century. It is used for drinking and wastewater treatment, air disinfection, the treatment of foods, as well as, for the disinfection of a myriad of home devices including toothbrushes, phones, and various electronic devices. While UV light can be a very efficacious germicidal agent, it is also mutagenic and carcinogenic. Thus, to date, its utilization has been limited by safety concerns.

In general, when UV irradiation has been used to disinfect food, air, or water, short wavelengths of UV light, typically in the UVC range of about wavelengths 240 to 280 nanometer, have been used. Such UV irradiation can be produced with low-pressure mercury lamps or Light Emitting Diodes (LEDs). Low-pressure mercury lamps can produce a range of UV wavelengths, ranging from UVA (about wavelengths 315 to 400 nanometer) to UVB (about wavelengths 280 to 315 nanometer) or to UVC. Commonly, mercury-vapor lamps or specialty LEDs emit UV radiation at around 254-260 nanometer, which may be harmful to humans and other life forms. For safety reasons, these mercury-vapor lamps and LEDs are typically shielded or in environments where exposure is limited. Thus, depending upon the environment, the utility of such lamps is limited or excluded.

### SUMMARY

According to various embodiments, improved disinfection methods, systems and devices that emit a narrow spectrum of far UV light to eliminate biological contaminants are provided. Systems and devices of embodiments of the present invention do not require shielding to protect users from harmful UV radiation.

According to embodiments, lighting fixtures emit far UV-C light. The lighting fixtures may be programmed or adjusted to only emit UV light within a narrow spectrum band for a specified period(s) of time. Preferably, these lighting fixtures only or primarily emit light within the spectrum of 200 nanometer-235 nanometer or 200 nanometer-230 nanometer wavelengths. Alternatively, these lighting fixtures can be adjusted to emit light within the spectrum of 200 nanometer-280 nanometer wavelengths to disinfect, or sanitize, a wide variety of surfaces.

According to embodiments, when conditions exist where humans may be present, light fixtures of the invention only or primarily emit light within the spectrum of 200 nanometer-230 nanometer wavelengths. Alternatively, according to embodiments, when humans are absent from the area to be treated, light fixtures of the invention may emit light within the broader spectrum of 200 nanometer-280 nanometer wavelengths.

According to embodiments, devices and systems may be adjusted or programmed to emit UV light continuously, intermittently for a specified period(s) of time, only when no movement (e.g. caused by a person's presence) is detected in an area, or a combination thereof. Additionally or alternatively, devices and systems of the invention may be adjusted or programmed to stop or reduce emitting UV light when movement is detected in a treatment area. For example, it may be desirable to continuously treat an area except when a human is present in the area or vary dosage to specific Threshold Limit Values or other preferences.

According to embodiments, devices and methods may detect movement include an ultrasonic, infrared (IR), or other type(s) or combinations of sensor(s) that can be used to determine whether an area is occupied. A variety of such sensors are known in the art and are suitable for use in devices of the invention.

According to embodiments, devices and methodsutilize far UV light, or radiation, that is within the spectrum of 200 nanometer-280 nanometer wavelengths to disinfect, or sanitize, a wide variety of surfaces. Preferably, these devices do not generate significant wavelengths of greater than 230 nanometer UV light outside of this range. Alternatively, according to embodiments, if these devices include a lamp or light source that emits wavelengths of UV light outside of this range then these devices could include a filter(s) that prevents or minimizes UV radiation outside of this range from being emanated into the environment. Importantly, exposure to UV light within the spectrum of 200 nanometer-230 nanometer wavelengths does not result in mutagenic or carcinogenic effects to human or animal cells. Thus, according to embodiments, devices and methods only or predominantly radiate UV light within the spectrum of 200 nanometer-230 nanometer wavelengths into an environment do not require shielding to be used safely.

According to an embodiment, a preferred lamp or light source is a krypton chloride lamp. Those of skill in the art will appreciate that alternative lamps and light sources exist. The choice of the type of lamp will be determined at least in part by availability, costs, type of power source, lamp size, and heat generated among other factors that are associated with the specific environment in which a device is to be used.

According to embodiments, devices include at least one light source that generates photons of at least one wavelength within the range of 200 nanometer to 280 nanometer. According to embodiments, at least one light source generates photons within the range of 200 nanometers to 230 nanometers. Photons may be directed through air and/or at a surface such that exposing the surface to the photons achieves at least a ninety percent kill of microorganisms on the surface. According to embodiments, wavelength ranges are between 205 nanometers and 210 nanometers or between 220 nanometers and 225 nanometers. According to embodiments, a light source generates photons that are predominantly at a wavelength of 207 nanometer or a wavelength of 222 nanometer.

According to embodiments, devices include at least one light source that generates ultraviolet (UV) light of at least one wavelength within a range of 200 nanometers to 230 or 235 nanometers, wherein the photons are directed through air or at a surface such that exposing the surface to the photons achieves at least a ninety percent kill of microorganisms on the surface. According to an embodiment a housing for the light source has an opening (e.g. a window) through which UV light is directed; potentially a timer; and electrical circuitry that can connect to a power source.

According to embodiments, devices may include a controller such as an on/off button. Alternatively, a controller may be separate from a device. For example, a device may be a ceiling fixture that is connected to a power source that is controlled by a wall switch or other controller including a smart phone or other IoT device(s).

According to embodiments, a housing of a device can include a fan, a vent(s), or a combination thereof. Such fans or vents may be suitable in devices that may experience excessive heat. Devices that include ventilation may be constructed such that extraneous UV light is not emitted unintentionally. For example, vents may be part of the opening through which UV light is intentionally directed; vents may be located in the housing so that any extraneous UV light is directed at the surface to which the device is attached; the housing may include an internal shield that blocks UV light from being emitted through a vent; or a combination thereof.

According to various embodiments, devices include light fixtures that are attached to a ceiling, wall, or other suitable surface. According to other embodiments, devices include light fixtures that are portable. Such portable devices may include a power source (e.g. a battery, capacitor, or other suitable power source known in the art) and a controller (e.g. an on/off switch). Alternatively, portable devices may include a cord or cable with which such devices may be attached to a power supply such as an AC wall socket. Portable devices may be temporarily installed or placed in an area to be disinfected. Those of skill in the art will recognize that such portable devices may remain positioned in an area for an extended period of time.

According to embodiments, devices and systems are pre-programmed and/or IoT (Internet of Things) compatible. For example, devices and systems may include Bluetooth®, WiFi, ultrasonic or RFID technology that can send data (e.g. motion detection) to a transceiver (via an antenna or wire) or alternatively receive instruction (e.g. when to emit UV light or for how long to emit UV light). Such systems and devices may be pre-programmed or re-programmed to emit UV light on different schedules as determined by the user.

According to other embodiments, devices and systems may omit IoT compatibility. Such devices may be preferred in certain types of environments where IoT compatibility is not desirable. Devices with or without IoT compatibility may be activated and deactivated manually with a switch that is located internal to the device or separate from the device, and connected to a power supply (e.g. a wall switch), or use a timer that may be set manually. The timer may be internal or external to the housing of the device.

Embodiments also provide systems for removing microorganisms from a surface, comprising at least one light source that generates ultraviolet (UV) light of at least one wavelength within the range of 200 nanometer to 280 nanometer, wherein the UV light is directed at the surface such that at least a ninety percent kill of selected microorganisms on the surface is achieved.

Embodiments further provide a means of determining distance from the device to a surface to be treated or to an inhabited area, wherein the inhabited area is an area that a human or animal may occupy. It is expected that the inhabited area is within the area that will be exposed to UV light when the targeted surface is treated. Advantageously, by determining the distance to an inhabited area, some embodiments of the invention allow the user to adjust a device so that it maximizes disinfection within regulatory guidelines or other restrictions for exposure to UV radiation.

Embodiments provide methods of disinfecting comprising exposing a surface to UV light within the range of 200 nanometer to 280 nanometer wavelengths, more preferably within the range of 200 nanometer to 230 nanometer, such that at least a ninety percent kill of selected microorganisms on the surface are killed by using the devices and systems described and disclosed herein.

Advantageously, any one embodiment may use elements disclosed with reference to other embodiments. Further, multiple or different elements may be used in any one embodiment. For example, according to an embodiment, the device includes a motion sensor, while devices of other embodiments do not. According to an embodiment, a plurality of devices are linked to one another such that when one device detects motion in an area undergoing disinfection, the system shuts off the lamps in all or some of other devices.

According to another embodiment, devices are mounted at different distances or angles from a surface(s) (e.g. over or along a stairwell) that is to be disinfected. According to an embodiment, devices may be adjusted to account for the different distances and angles present in the area so that any one device can be turned on for an appropriate period of time to treat a specific section of the total area to be treated or to achieve a desired result.

Those of skill in the art will appreciate that, while embodiments of the invention are described with particular reference to the safety of humans, similar principles apply to the use of the similar devices in the presence of animals that are susceptible to harm by exposure to UV radiation, and that appropriate safety procedures can be tailored to the needs of the particular types of animals that may be present in areas where the devices are to be used. Furthermore, it is within the ability of a person having ordinary skill in the art, in view of the present disclosure, together with other known principles, to devise such procedures or adapt procedures that are disclosed herein.

According to an embodiment, a germicidal lamp includes one or more light sources that predominantly emits wavelengths of ultraviolet (UV) light within the range of 200 nanometer to 235 nanometer such that at least 90% of selected microorganisms within a defined treatment area are killed or deactivated, a timer, electrical circuitry, and a housing for the one or more light sources, timer, and electrical circuitry, the housing having a front cover and a back cover that are fastened to each other, one or both of the front cover and back cover including one or more extensions and/or dividers configured to hold the one or more light sources, timer, and electrical circuitry in place.

According to an embodiment, a method for eliminating selected microorganisms in a defined treatment area includes exposing the treatment area to one or more light sources that only emits wavelengths of ultraviolet (UV) light within the range of 200 nanometer to 230 nanometer for a selected period such that at least a 90% of selected microorganisms in the treatment area are killed or inactivated.

According to an embodiment, a method of disinfecting includes exposing a treatment area to one or more light sources that only emits wavelengths of ultraviolet (UV) light within the range of 200 nanometer to 230 nanometer such that at least 90% of microorganisms on the surface are killed.

According to an embodiment, an ultraviolet luminaire includes a housing, including a front housing defining an aperture and a back housing, the front and back housings being configured to contain electrical components of an ultraviolet luminaire and t least one mounting point operatively coupled to the back housing. An ultraviolet emitter is mounted within the housing and configured to project, through the aperture defined in the front housing, ultraviolet light energy between 200 and 235 nanometers wavelength.

According to an embodiment, an ultraviolet luminaire includes a housing including a front housing and a back housing, the front and back housings being configured to contain electrical components of an ultraviolet luminaire, an ultraviolet emitter positioned within the housing, an electronic controller contained by the housing, and a light shield, formed in one or both of the front housing and back housing and configured to substantially prevent heat and ultraviolet light produced by the ultraviolet emitter from reaching the electronic controller.

According to an embodiment, a germicidal lamp system configured to output ultraviolet illumination into a defined treatment area at least intermittently occupied by one or more humans includes a light emitter configured to output ultraviolet light into a defined treatment area, a sensor configured to detect the presence of a human in the defined treatment area, and an electrical controller operatively coupled to the light emitter and the sensor, and configured to control the light emitter responsive to a signal from the sensor indicating the presence of a human in the treatment area.

According to an embodiment, a portable germicidal lamp system includes a housing, a UV-C light source disposed in the housing and configured to output ultraviolet light into a space at least intermittently occupied by one or more persons, power supply configured to provide AC waveform to the UV-C light source, and an exposed interface configured to receive actuation to couple the power supply to the UV-C light source.

According to an embodiment, an auto-ranging UV-C source includes a housing, an excimer light source disposed in the housing and configured to output UV-C light to kill pathogens in an exposure space having a centerline and extending away from the housing, an electronic controller disposed at least partially in the housing and operatively coupled to the excimer light source, and a distance sensor operatively coupled to or integrated with the electronic controller, the distance sensor being aligned to the centerline of the exposure space and configured to measure one or more distances from the housing to a surface bounding the exposure space, a person in the exposure space, or to the surface and the person. The electronic controller is configured to receive data corresponding to the one or more distances from the distance sensor and to control the excimer light source to control a UV-C power intensity according to a pre-determined exposure limit at the person, the surface, or the person and the surface.

According to an embodiment, an ultraviolet illuminator and/or system includes a krypton-halogen emission source configured to output far ultraviolet-C (far UV-C) light into an illuminated region and an electronic controller operatively coupled to the krypton-halogen emission source. The electronic controller may include an krypton-halogen emission source driver configured to control the krypton-halogen emission source. The electronic controller may include a logic circuit operatively coupled to the krypton-halogen emission source driver, and configured to control the krypton-halogen emission source driver, and a communication interface operatively coupled to the logic circuit. The communication interface may be configured for bidirectional communication according to a digital interface protocol. In an embodiment, the electronic controller is configured to control the krypton-halogen emission source to operate according to data received by the communication interface. In an embodiment, the far UV-C light is characterized by a passband that includes wavelengths between 200 nanometers and 230 nanometers wavelength. In another embodiment, the far UV-C light is characterized by a passband that includes wavelengths between 200 nanometers and 235 nanometers wavelength. In an embodiment, the ultraviolet illuminator includes one or more sensors operatively coupled to the logic circuit and configured to sense a parameter corresponding to the illuminated region. The logic circuit may select an operation parameter for the krypton-halogen emission source responsive to the sensed condition.

According to an embodiment, a computer method for controlling far UV-C illumination of an region may include reading first control data including a schedule for far UV-C illumination with a first electronic controller disposed in a first housing, inferring a state of an exception condition corresponding to a first illuminated region, and driving, with the first electronic controller, a krypton-halogen emission source disposed in the first housing to output far UV-C light into the first illuminated region according to whether the exception condition is negative or positive. While maintaining driving of the krypton-halogen emission source, the method includes looping to again read the first control data, inferring the state of the exception condition, and continuing or changing the output of the krypton-halogen emission source according to the most recently inferred exception condition. The method may include initiating an ultraviolet illuminator and the electronic controller by transmitting the control data to the first electronic controller from a computing device.

According to an embodiment, an excimer lamp includes a quartz glass tube holding krypton and a halogen that form a gas characterized by an excimer discharge energy including ultraviolet light emission between at least a portion of a range delimited by 200 nanometers and 235 nanometers wavelength, which is invisible to a human eye. An electrode pair is mechanically supported by or adjacent to the quartz glass tube configured to, when an alternating voltage is applied, capacitively charge the gas to form a Kr—Cl or Kr—Br excited complex, whereupon the gas undergoes the excimer discharge when the krypton and halogen dissociate. An optical diffuser disposed in an illumination path optically aligned between the quartz glass tube and an illuminated region, suitable for human occupation, converts the excimer discharge to an emission pattern approaching Lambertian emission. According to an embodiment, the optical diffuser includes poly-tetra-fluoro-ethylene (PTFE).

According to an embodiment, a far ultraviolet illuminator includes a housing defining an illumination aperture, an electronic circuit disposed in the housing and configured to output an alternating current to two or more electrical connection points for supply to an excimer lamp electrode pair, a mechanical coupler configured to hold the excimer lamp at least partially within a volume defined by the housing and in alignment with the illumination aperture, and an optical diffuser configured to convert far ultraviolet illumination emitted from the excimer lamp and illumination aperture to diffuse illumination such as Lambertian emission.

According to an embodiment, an excimer illumination system includes a system housing defining a housing illumination aperture, an excimer drive circuit disposed in the system housing and including at least two lamp drive electrical connection points, the excimer drive circuit being configured to output, to the at least two lamp drive electrical connection points, an alternating current for driving an excimer lamp. A physical mounting structure may be provided for receiving and holding the excimer lamp such that the excimer lamp is at least partially disposed inside the system housing. At least a portion of the excimer lamp is configured to be removed and replaced by the user at a location where the excimer illumination system is installed.

According to an embodiment, an excimer lamp includes a quartz glass tube holding krypton and a halogen that form a gas characterized by an excimer discharge energy including ultraviolet light emission between at least a portion of a range delimited by 200 nanometers and 235 nanometers wavelength, an electrode pair mechanically supported by or adjacent to the quartz glass tube configured to, when an alternating voltage is applied, capacitively charge the gas, whereupon the gas undergoes the excimer discharge, an excimer lamp housing supporting the quartz glass tube and the electrodes, and at least one connector operatively coupled to the electrode pair, the at least one connector being configured for reversibly coupling to a circuit in a far ultraviolet illumination system. The excimer lamp may be replaceable at a location where an excimer illumination system is installed.

According to an embodiment, an excimer lamp includes a quartz glass tube holding krypton and a halogen that form a gas characterized by an excimer discharge energy including ultraviolet light emission between at least a portion of a range delimited by 200 nanometers and 235 nanometers wavelength, an electrode pair mechanically supported by or adjacent to the quartz glass tube configured to, when an alternating voltage is applied, capacitively charge the gas, whereupon the gas undergoes the excimer discharge, a lamp housing supporting the quartz glass tube and the electrodes, and at least one connector operatively coupled to the electrode pair, the at least one connector being configured for reversibly coupling to a circuit in a far ultraviolet illumination system. The excimer lamp may be replaceable at a location where an excimer illumination system is installed.

According to an embodiment, an excimer lamp includes a quartz bulb holding a halogen and krypton gas, at least two electrodes configured to at least partially support the quartz bulb, an electrically insulative lamp housing configured to physically support the at least two electrodes. The electrically insulative lamp housing includes a back surface and at least one side surface defining a perimeter of an internal lamp volume, the electrically insulative lamp housing and the at least one side surface defining a lamp aperture disposed opposite to the back surface of the lamp housing. Two or more electrical conductors operatively coupled to respective electrodes extend through one or more holes through the electrically insulative lamp housing. The two electrical conductors are configured to convey power for electrically energizing the at least two electrodes, causing dielectric barrier discharge inside the quartz bulb and through the halogen and krypton gas to cause the halogen and krypton gas to undergo excimer discharge. In an embodiment, the excimer lamp is replaceable as a unit. In an embodiment, the quartz bulb is replaceable.

According to an embodiment, a kit for user replacement of a quartz bulb containing krypton and a halogen includes a replacement quartz bulb, instructions for replacing the quartz bulb by snapping the quartz bulb into a pair of electrodes akin to a fuse clip, and a planar material selected for preventing the human fingers from touching an outside surface of the quartz bulb during installation.

According to an embodiment, an ultraviolet illuminator with ozone mitigation includes a housing defining an interior volume, an electronic controller and a far UVC source operatively coupled together and disposed in the interior volume of the housing, the far UVC source being configured to emit far UVC illumination, and a window disposed to pass at least a portion of the far UVC illumination output by the far UVC illumination source into an external illuminated region. At least one fan is configured to draw air from the illuminated region into the interior volume of the housing. At least one ozone filter is arranged to filter the air drawn from the illuminated region prior to discharge of the air to an exterior region different than the illuminated region. The at least one fan and at least one ozone filter are configured to cooperate to reduce a concentration of ozone in the illuminated region formed by interaction of oxygen with the far UVC illumination.

According to an embodiment, an ultraviolet illuminator includes a housing defining an interior volume configured as an air plenum. A far UVC source is operatively coupled to the housing and configured to output far UVC illumination into an illuminated region. One or more air receiving structures are configured to convey air preferentially from the illuminated region into the interior volume. A fitting is configured to couple to an air duct for conveying air from the interior volume to the air duct. The air duct may be operatively coupled to a blower and/or an ozone filter. The blower may be configured to convey the air through the one or more air receiving structures from the illuminated region, through the interior volume, through the fitting, through the air duct, and through the ozone filter such that ozone in the air is converted to molecular oxygen.

According to an embodiment, a compliant far UVC light illuminator includes a far UVC light source and light source driver circuit configured to initiate an arc in the far UVC light source and reduce energy delivered to the far UVC light source within less than 10 seconds. Initial ozone output corresponding to start-up of far UVC emission is thereby minimized.

According to an embodiment, a far UVC illuminator includes a diffuser disposed in an illumination path from the far UVC illuminator, the diffuser being selected to reduce ozone production by up to 49%.

According to an embodiment, a far UVC illuminator product line includes at least one first stock-keeping unit (SKU) that produces far UVC illumination while meeting ozone concentration limits corresponding to UL2998. A far UVC product line also includes at least one second SKU that produces far UVC illumination while meeting ozone concentration limits corresponding to UL867 and not meeting ozone concentration limits corresponding to UL2998.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description. Further, while specific advantages of the invention are detailed herein, various embodiments may include some, none, or all of these enumerated advantages.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description presented herein. Unless specifically noted, articles depicted in the drawings are not necessarily drawn to scale.

FIG. 1 is a front exterior of a lighting fixture, according to an embodiment.

FIG. 2 is a back exterior of the lighting fixture of FIG. 1, according to an embodiment.

FIG. 3 is an interior view of front cover of the lighting fixture of FIGS. 1 and 2, according to an embodiment.

FIG. 4 is an interior view of back cover of the lighting fixture of FIGS. 1-3, according to an embodiment.

FIG. 5 is an interior view of the lighting fixture of FIGS. 1-4, according to an embodiment.

FIG. 6 is a diagrammatic view of a germicidal lamp system, according to an embodiment.

FIG. 7 is a diagrammatic view of a germicidal lamp system, according to another embodiment.

FIG. 8 is a perspective view of a portable germicidal lamp 800, according to an embodiment.

FIG. 9 is a diagrammatic view of a portable germicidal lamp 900, according to another embodiment.

FIG. 10 is a chart 1000 showing levels of UV intensity or strength at various output levels, according to an embodiment.

### DETAILED DESCRIPTION

In the following detailed description, reference is made to the accompanying drawings, which form a part hereof. In the drawings, similar symbols typically identify similar components, unless context dictates otherwise. Other embodiments may be used and/or other changes may be made without departing from the spirit or scope of the disclosure.

Embodiments of the invention provide a variety of devices that emit far UV radiation that is directed through air and towards one or more surfaces, which it is desirable to disinfect from selected microorganisms or allergens.

Each of the devices includes one or more UV light sources. These UV light sources can be a krypton chloride lamp, krypton bromide lamp, an excimer lamp, a low-pressure, mercury-arc germicidal lamp, a dual annulus lamp, a light emitting diode (LED), or a combination thereof. According to an embodiment, preferred light sources are krypton chloride lamps. In certain embodiments, plasma or xenon systems that emit UV radiation within the range of range of 200 nanometer to 280 nanometer wavelengths, or 200 nanometer to 230 nanometer wavelengths, also may be included in devices of the invention.

When devices, systems, and methods according to various embodiments of the invention are operated, it is expected that at least 90%, 92%, 95%, 96%, 97%, 98%, 99%, 99.9%, 99.99%, 99.999%, 99.9999% or even 99.99999% or more of all or selected microorganisms or allergens in air or on the exposed surfaces will be killed or effectively rendered inert. Those of skill in the art will recognize that the percentage of kill and the amount of time that it takes to achieve a desired mortality is, in part, dependent upon the distance between the material and light source(s). Thus, the skilled artisan will understand that both the distance from the light source and time of exposure can be adjusted to achieve a desired result. Similarly, the skilled artisan will understand that including multiple light sources in a system or emitting more than one wavelength of photons from a device or system during operation will also effect the percentage of microorganisms killed and the amount of time required to achieve a desired mortality.

According to an embodiment, a specific area may be identified for treatment with UV light, such as, e.g., a room or portion of a room, a food preparation surface, a work surface, a building entryway, etc. The treatment area may also include the space—i.e., air—between the UV light source and surface(s) to be treated. According to an embodiment, a treatment area may include or be limited to a region corresponding to the air breathed by persons standing and/or sitting within a defined space. This may be especially effective for killing viruses on particles (e.g., droplets and/or aerosol) in a shared breathing airspace. According to an embodiment a rate of UV photon delivery may be selected to reduce, significantly reduce, or effectively prevent airborne transmission of selected pathogens. Where the effectiveness of a particular embodiment or system is discussed herein, it is with respect to such a treatment area, even though UV light produced for the purpose of treatment may not be entirely confined to within the area.

In some cases, it may be desirable to kill most or substantially all microorganisms present within a particular environment, or on a particular surface, i.e., to produce a near sterile condition. However, in other cases, it may be sufficient to kill only those microorganisms that are harmful or detrimental or otherwise of interest under the particular circumstances, while other, benign or harmless microorganisms can be safely ignored. In such cases, the exposure time, intensity, and wavelengths of the UV radiation can be selected to achieve a desired kill rate of the selected microorganisms, even if a kill rate among some harmless microorganisms is lower.

According to an embodiment, light sources used emit UV radiation substantially within the range of 200 nanometer to 230 nanometer wavelengths. In embodiments in which a light source(s) generates UV radiation greater than 230 nanometer wavelengths, then one or more filters can be included such that over 99% of UV radiation greater than 230 nanometer wavelengths is absorbed by the filter(s) and is not emitted into the surrounding environment. For example, a coating on a reflector, planar surface, interior of the housing, or a combination thereof may be used to absorb harmful wavelengths. According to an embodiment, a filter(s) is directly attached or adjacent to the light source so that only UV radiation within the range of 200 nanometer to 230 nanometer wavelengths is emitted into the environment from the light source.

It is envisioned that embodiments can be used for a wide variety of purposes in multiple industries. For example, A UV germicidal lamp may be incorporated into safety protocols associated with the use or manufacture of electronics, computers, telephones, mobile devices, and the like. According to various embodiments, UV germicidal lamps are provided to disinfect appliances, countertops, laundry, textiles, floors, rugs, dishes, utensils and their holders, vacuum cleaners (e.g. Roomba®), and food storage containers.

According to an embodiment, a lighting fixture is provided that may be used to disinfect or sanitize areas without the aid of other cleansers. For example, such a device can be used in the place of sinks in either public or household environments. Advantageously, less waste is generated in such environments, which can assist in reducing the transmission of disease-causing organisms.

According to some embodiments, UV germicidal lamps may be used in food processing or preparation. For example, the claimed systems and devices can be used to disinfect work surfaces for meat and poultry, as well as the equipment used to prepare or transport meat, poultry, or other food products. Embodiments of the invention provide improved methods for producing a dry, chemical free, disinfection of surfaces.

The invention can be configured to selectively kill or render inert at least one microorganism on a surface, while substantially avoiding harm to human or animal cells. The light source of the far UV radiation can include a krypton-bromine lamp, a krypton-chloride lamp excilamp, or other UV light emitting lamp. According to an embodiment, the light source is a krypton-chloride lamp that emits UV wavelengths between 200 nanometer and 280 nanometer, and more preferably between 200 nanometer and 230 nanometer. According to various embodiments, a narrow band of wavelengths of far UV radiation can be emitted from a device. For example, in one embodiment, wavelengths can be emitted between 205 nanometer and 210 nanometer or, in another embodiment, between 220 nanometer and 225 nanometer. According to some preferred embodiments, the UV wavelengths that are emitted are predominantly at 207 nanometer (KrBr*) or 222 nanometer (KrCl*).

In some embodiments, exemplary features can be included (e.g., spectrum filtering elements such as multilayer dielectric filters or chemical filters or different chemistries within the lamps themselves) to remove unwanted wavelengths, or those wavelengths that are outside of the preferable range of wavelengths. For example, absorption and/or reflective elements can be provided between the lamp and the irradiated surface to filter unwanted wavelengths, such as, e.g., a band-pass filter or a long-wavelength blocking filter.

Embodiments of the invention that incorporate multiple devices also can be used to disinfect large areas having multiple types of surfaces present. Advantageously, Embodiments of the invention can be configured to conform to areas of various sizes and shapes. Those of skill in the art will understand that the number of devices required for any area will depend, at least in part, on the distance of the device(s) from the element that is to be treated.

Distance plays a factor in the efficacy of UV light as a disinfectant. Following the inverse square law, the strength of UV radiation will decrease as it gets further away from its source. For example, UV light will have one quarter of its power (per unit of area) when it is twice the distance from its source that it had at the original reference point. This relationship limits how far a single source of UV light will be effective before it is too weak to provide adequate disinfection. The UV dose is the product of UV intensity [I] (expressed as energy per unit surface area) and exposure time [T]. Therefore: DOSE=I×T. This dose, sometimes referred to as fluence, is commonly expressed as millijoules per square centimeter (mJ/cm²). The units “J/m²” are used in most parts of the world except for North America, where “mJ/cm²” are used. Thus, for any given configuration of a device disclosed herein, it is possible to calculate the UV light intensity that will be achieved.

According to an embodiment, in systems that are used in the presence of humans, the cumulative exposure to UV light from a device(s) is controlled so as not to exceed the regulatory recommended amount. For example, currently it is recommended that the cumulative exposure of a person to UV light during a work period does not exceeds 22 mJ/cm². A “work period” is variously interpreted as 8 hours to 24 hours, depending upon the authority being referenced. According to various embodiments, exposure may be controlled by the use of timers that regulate a period during which a device is in operation or a duty cycle of operation, the use of motion detectors to deactivate UV germicidal lamps while humans are present or within a selected range of the devices, the use of filters to limit the wavelengths of light that are emitted, the selection of the output intensity of the devices, etc.

Further, the reduction of many different types of microorganisms is classified using a logarithmic scale and is known. For example, a single log reduction is a 90% reduction of organisms; a two log reduction is a 99% reduction of organisms; a three log reduction is a 99.9% reduction of organisms; and a seven log reduction is up to a 99.99999% reduction of organisms. Thus, for any particular configuration of a device of one or another embodiment, it is possible to determine the amount of time of exposure that is required to achieve the desired kill rate for various microorganisms based on the amount UV light intensity that can be achieved at the point of contact of the surface that is to be treated, and the sensitivity of the different microorganisms to the particular wavelengths of UV light employed.

By knowing the various distances between the devices and areas to be treated, the types of surfaces to be treated, and the preferred amount(s) of time of exposure, the skilled artisan can calculate the amount of overlap and number of devices that will be needed for a particular system to kill or render inert the selected microbes or allergens in the treated area. Further, the skilled artisan will understand that greater or lesser amounts of exposure to UV light may be preferred in some instances to kill or render inert different microbes or allergens or for the ease of mind of users of the area.

Unless defined otherwise, all technical and scientific terms used herein have the same meaning as is commonly understood by one of skill in the art to which this invention belongs at the time of filing. Terminology used herein is for the purpose of describing particular embodiments of the invention and is not intended to be limiting. The meaning and scope of terms should be clear; however, in the event of any latent ambiguity, definitions provided herein take precedent over any dictionary or extrinsic definition. Further, unless otherwise required by context, singular terms shall include pluralities and plural terms shall include the singular unless the content clearly dictates otherwise. Herein, the use of “or” means “and/or” unless stated otherwise. Furthermore, the use of the term “including”, as well as other forms such as “includes” and “included” is not limiting. As used herein, “each” refers to each member of a set or each member of a subset of a set. All patents and publications referred to herein are incorporated by reference herein.

It should be appreciated by those of skill in the art that the techniques disclosed in the following examples represent techniques discovered by the inventors to function well in particular applications, and thus can be considered to constitute preferred modes of practice for some applications. However, those of skill in the art should, in light of the present disclosure, appreciate that many changes can be made in the specific embodiments which are disclosed and still obtain a like or similar result without departing from the spirit and scope of the invention. Thus, the present invention should not be limited to the exemplary embodiments and techniques illustrated herein.

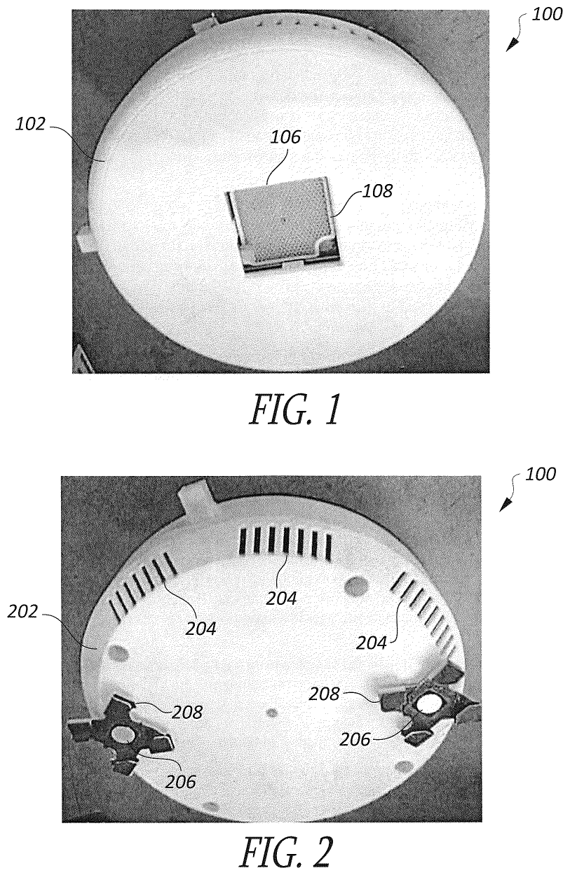

FIGS. 1-5 show an exemplary lighting fixture 100, or luminaire, for attachment to a flat surface, according to an embodiment. As shown in FIG. 1, the fixture 100 includes a front housing 102 and a back housing 202 (see FIG. 2) that are configured to be coupled together to form a complete housing of the fixture 100. The front housing 102 has an opening or aperture 106 through which UV light can be emitted. Through this opening 106, a krypton-chloride, krypton-bromide, or other exciplex moiety lamp 108 configured to output at least UVC radiation at wavelengths described herein is disposed.

The exterior of the back housing 202 is shown in FIG. 2 and includes multiple vents 204 through which air can move passively are visible around an outer perimeter of the back housing 202. In this example, any UV light that may be emitted through these vents is directed towards a surface to which the fixture may be attached.

In this example, the back housing 202 includes mounting points 206 to which respective fasteners 208 are operatively coupled, which can be used to install the fixture 100 to a surface. The fasteners 206 shown in FIG. 2 are in the form of clips configured to engage the grid rails (also referred to as runners, tees, tracks, etc.) of a standard drop ceiling (aka suspended ceiling). These clips 208 enable the temporary or permanent attachment of the fixture 100, or multiple such fixtures, to a drop ceiling. According to various embodiments, a wide variety of clips, screws, tapes, Velcro, glue and other fasteners that are known in the art can be substituted for the clips 208 shown in FIG. 2. It is only necessary that the fasteners are able to fix the device to the desired surface securely. Such fasteners may be configured to affix the fixture 100 either permanently or temporarily to a surface. According to an embodiment, the mounting points 206 may be configured to accommodate a selected type of fastener. For example, in embodiments in which the fixture 100 is designed to be mounted to a surface with a hook-and-loop fastener, the mounting points 206 may include relatively broad, flat surfaces to which adhesive-backed hook or loop fastener strips can be coupled.

According to other embodiments, the lighting fixture 100 can be configured to be mounted to other surfaces. For example, in one embodiment, the back housing 202 includes a mounting point with an aperture tapped with a ¼×20 thread. Many tripods and stands configured for use with photographic and audio equipment include mounting brackets with captured ¼×20 machine screws configured to engage corresponding threaded apertures in cameras, flash systems, microphones, loudspeakers, etc. The tapped aperture of the present embodiment enables a user to mount the lighting fixture 100 to a camera tripod or loudspeaker speaker stand, for temporary use in a selected location.

According to another embodiment, the back housing 202 includes a loudspeaker keyhole mounting point (not shown) for mounting the lighting fixture 100 to, e.g., a wall-mounted loudspeaker bracket, and provide ultraviolet illumination of a treatment area from a wall.

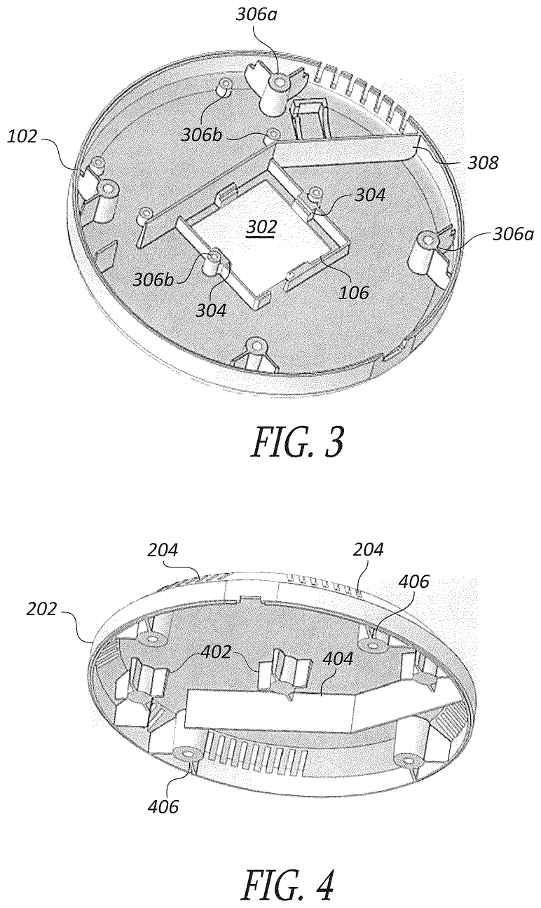

The interiors of the front and back housings 102, 202 are illustrated in FIGS. 3 and 4, respectively. As shown in FIG. 3, the opening 106 in the front housing 102 can be covered by a transparent filter. Notches 304 are provided, configured to receive the lamp 108. In this illustration, the interior of the front housing 102 includes multiple bores 306 of various sizes, and divider 308. Here, larger bores 306a are provided for screws that attach the front housing 102 to the back housing 202. Smaller bores 306b are provided to attach internal components of the light fixture 100 to the interior of the front housing.

As shown in FIG. 4, the interior of the back housing 202 includes various extensions 402 and a divider 404 that assist in securing the internal parts of the fixture 100 in place. The interior of the back housing 202 also includes standoffs 406 to assist in securing the back housing 202 to the front housing 102. Those of skill in the art will appreciate that any or all of the various bores may be replaced by suitable extensions, notches, or other features in the housing. The choice depends, at least in part, upon preferred manufacturing methods and the availability and configuration of suitable internal parts. Similarly, the configurations and choices of extensions and dividers to hold the internal parts in place will depend, at least in part, upon the selection and configuration of the various internal parts.

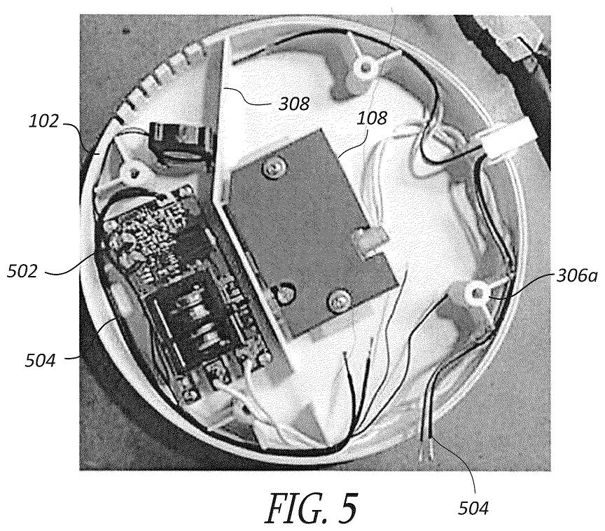

An example of one configuration of the internal parts is shown in FIG. 5, according to an embodiment. Control circuitry 502, the lamp 108 (as shown from the back), and the necessary connectors 504 are shown. According to an embodiment, the divider 308 of the front housing 102 and the divider 404 of the back housing 202 are configured to cooperate, when the front and back housings are coupled together, to function as a shield, to substantially prevent heat and ultraviolet light from the lamp 108 from reaching the control circuitry 502.

According to an embodiment, the control circuitry 502 includes a timer. According to another embodiment, the control circuitry 502 includes a processor and memory for programmable operation of the fixture 100. According to further embodiments, the control circuitry may include one or more of a receiver configured to receive programming input, input from remote sensors, and syncing data from other similar fixtures, to receive user-initiated controls, and to interface with WiFi, Bluetooth, etc.; circuitry for regulating an intensity or duty cycle of the lamp 108, etc.

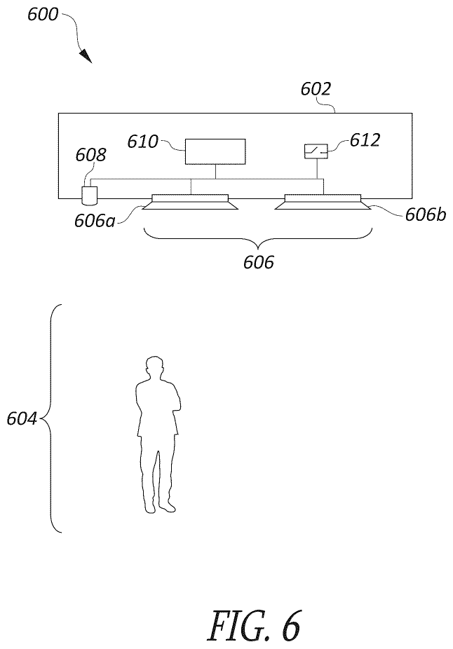

FIG. 6 is a diagrammatic view of a germicidal lamp system 600, according to an embodiment. The lamp system 600 includes a lighting fixture 602 positioned so as to output ultraviolet light and illuminate a treatment area 604 that is at least intermittently occupied by one or more humans. The lighting fixture 602 includes a light emitter 606 configured to output ultraviolet light, a sensor 608 configured to detect the presence of a human in the treatment area, and an electrical controller 610 operatively coupled to the light emitter and the sensor, and to control the light emitter responsive to a signal from the sensor indicating the presence of a human in the treatment area. According to an alternative embodiment, the controller 610 may separate from the fixture, and may be positioned near the lighting fixture 602 or at some distance therefrom. According to an embodiment, the light emitter is configured to output ultraviolet light of between 200 nanometers and 280 nanometers wavelength during normal operation.

According to an embodiment, the lamp system 600 includes a power switch 612 operatively coupled to the electrical controller 610.

According to an embodiment, the power switch 612 is formed as a portion of the electrical controller 610. According to another embodiment, the power switch 612 may be a separate component positioned outside the controller 610, and may be accessible to a user, or, alternatively, may be controlled exclusively by the controller.

In another embodiment, the power switch 612 may be controlled exclusively by the controller 610, while a second switch (not shown in detail) is positioned for access by a user. In this embodiment, the controller 610 may be configured to control or manage various aspects of operation of the light emitter 606, at least in part via control of the power switch 612, while the second switch enables the user to power up or down the entire germicidal lamp system 600, including the controller.

According to an embodiment, the power switch 612 may include a momentary switch by which a data signal to the electrical controller 610 initiates a power up of the electrical controller. The power switch 612 may also or alternatively be configured to prevent a data signal from powering up the electrical controller 610 while the power switch is switched off.

According to another embodiment, the power switch 612 may be configured to prevent the electrical controller 610 from energizing the light emitter 606 while the power switch is switched off.

According to an embodiment, the power switch 612 may include a momentary switch by which a data signal to the electrical controller 610 initiates a power up of the electrical controller and also a power down of the controller. For example, the controller 610 may be configured, while powered down, to power up upon receipt of a data signal via the switch 612 and, while powered up, to power down upon receipt of the same data signal via the switch. Alternatively, the switch 612 may be configured to transmit separate power up and power down signals to the electrical controller 610.

According to an embodiment, the electrical controller 610 may be configured to provide power to the light emitter 606, and to remove power from the light emitter while a signal from the sensor 608 indicates the presence of a human in the treatment area 604. The controller may be configured to control the light emitter 606 via the power switch 612, or alternatively may include an emitter control circuit by which the controller is configured to control the light emitter.

According to an embodiment, the sensor 608 may include a single sensor or type of sensor, or may alternatively include a plurality of sensors. For example, the sensor 608 may include one or more ultrasonic sensors, infrared sensors, and/or optical sensors. The sensor 608 may include one or more sensors configured to identify specific individuals. For example, the sensor 608 may include an RFID reader configured to identify RFID badges carried by individuals who are authorized to enter the treatment area.

According to further embodiments, the electrical controller 606 may be configured, while a signal from the sensor 608 indicates the presence of a human in the treatment area 604, to control the light emitter 606 to operate in a safe mode. Operation in the safe mode may include, operating at a reduced intensity, operating within or more selected wavelength ranges, and/or limiting a maximum duration of operation in the safe mode over a selected period, e.g., 24 hours.

For example, while operating in the safe mode, the light emitter 606 may be controlled to output not more than 479 millijoules per square centimeter at 222 nanometers wavelength, per day, while any particular human is detected in the treatment area. In other words, the controller 610 may be configured to identify individual humans that enter the treatment area, to track the total time that each human spends in the treatment area while the germicidal lamp system 600 is in operation, and to limit each human to a selected maximum exposure to the illumination of the light emitter over in a single day, or over a 24-hour period. The controller 610 may be configured to remove power from the light emitter 606 while any human who has received a maximum allowable daily exposure is detected in the treatment area 604.

According to an embodiment, the light emitter 606 may be controlled to output ultraviolet illumination only between 200 nanometers and 235 nanometers wavelength, or between 200 and 230 nanometers wavelength, while in operating in safe mode, i.e., while a human is detected in the treatment area.

According to an embodiment, the light emitter 606 includes a first light emitter 606a configured to output ultraviolet illumination between 200 nanometers and 230 or 235 nanometers wavelength, and a second light emitter 606b configured to output ultraviolet illumination between 200 nanometers and 280 nanometers wavelength. The electrical controller 610 may be configured to control both the first and the second light emitters 606a, 606b to operate during normal operation, and to control the second light emitter to shut down while a signal from the sensor 608 indicates the presence of a human in the treatment area 604. Alternatively, the electrical controller 610 may be configured to control only the second light emitter 606b to operate during normal operation, and to control the second light emitter to shut down and control the first light emitter 606a to power up while a signal from the sensor 608 indicates the presence of a human in the treatment area 604.

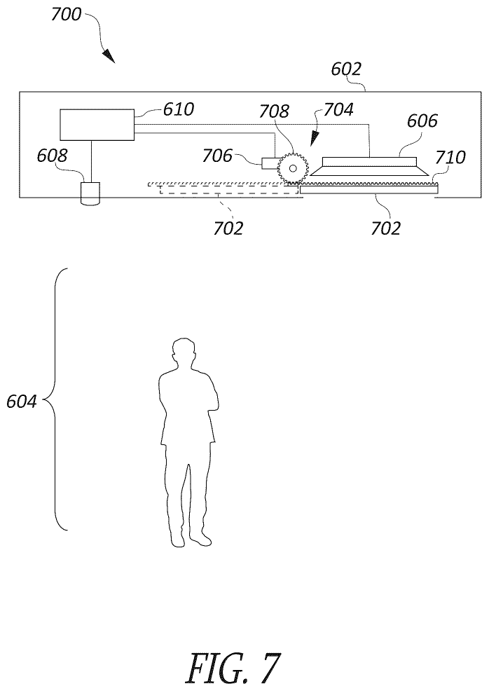

FIG. 7 is a diagrammatic view of a germicidal lamp system 700, according to an embodiment. The lamp system 700 includes a lighting fixture 602 that includes a light emitter 606, a sensor 608, and an electrical controller 610, as described with reference to previous embodiments. The lamp system 700 further includes a wavelength filter 702 configured to block selected wavelengths of light, and further configured to be selectively interposed between the light emitter 608 and the treatment area 604. An actuator 704 is operatively coupled to the wavelength filter 702 and to the electrical controller 610. The electrical controller 610 is configured to control the actuator 704 to interpose the wavelength filter 702 between the light emitter 606 and the treatment area 604 while a signal from the sensor 608 indicates the presence of a human in the treatment area.

According to an embodiment, the wavelength filter 702 is configured to block ultraviolet light between at least 236 nanometers and 280 nanometers wavelength. According to another embodiment, the wavelength filter 702 is configured to block ultraviolet light between 231 nanometers and 280 nanometers wavelength.

According to an embodiment, the wavelength filter 702 comprises a dielectric stack filter.

According to an embodiment, the wavelength filter 702 may include a band-pass filter, or alternatively a long-wavelength blocking filter.

According to an embodiment, the actuator 704 comprises a motor 706 and a linkage 708 operatively coupled to a rack 710 captured by grooves in a housing (not shown in detail). According to an embodiment, the electrical controller 610 controls the motor 706 to cause the rack 710 to slide in front of the ultraviolet light emitter 608 while a human is sensed in the treatment area 604 and controls the motor 706 to cause the rack 710 to slide away from in front of the light emitter 606 for at least a portion of time when a human is not sensed in the treatment area 604.

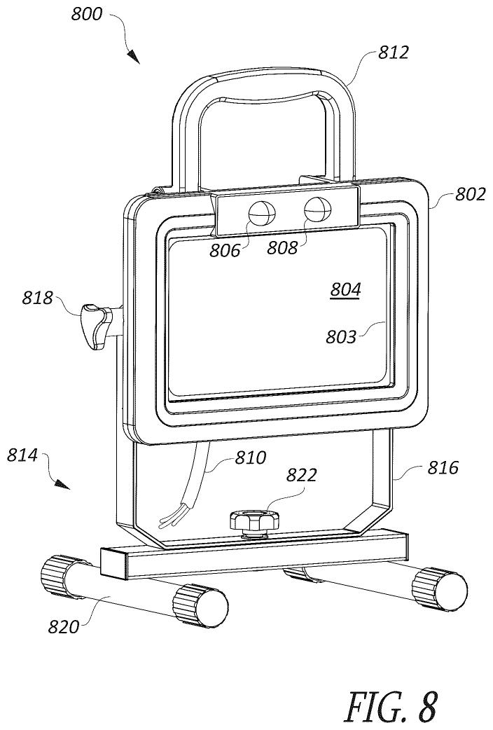

FIG. 8 is a perspective view of a portable germicidal lamp 800, according to an embodiment. The portable lamp 800 includes a housing 802 with an aperture 803 and an ultraviolet light emitter 804 of a type previously described, and in particular, a light emitter 804 capable of producing ultraviolet light within a range of between 200 nanometers and 235 nanometers wavelength. First and second sensors 806, 808 are mounted into a front face of the housing 802. The first sensor 806 is configured to detect the presence of a human in a defined treatment area. The second sensor 808 is configured to determine or measure a distance from the sensor to a target surface. Although not shown in FIG. 8. the portable germicidal lamp 800 further includes an electrical controller, substantially as described with reference to previous embodiments. The controller is operatively coupled to the light emitter 804 and the first and second sensors 806, 808. Electrical power for the lamp 800 is provided via a cable 810, which may be provided with a plug configured to mechanically and electrically couple with a standard electrical outlet. According to another embodiment, the portable germicidal lamp 800 is provided with an internal electrical battery, either in place of or in addition to the cable 810.

According to an embodiment, the housing includes a handle 812 for convenience in transporting the device. Additionally, a support structure 814 is provided to enable a user to select any of a number of options for positioning the portable germicidal lamp 800, as occasion requires. For example, a support frame 816 is coupled to the housing 802 via a pair of first fasteners 818 which permit the housing to rotate about the first fasteners, relative to the support frame and to be fixed at an angle selected by the user. A base 820 is in turn coupled to the support frame 816 via a second fastener 822, which permits the housing to rotate about the second fastener, relative to the support frame and to be fixed at an angle selected by the user. The support structure 814 may be configured by the user to safely and securely support the portable germicidal lamp 800 on a substantially horizontal surface in any of a wide range of orientations relative to the underlying surface.

According to various embodiments, the base 820 may be separated from the support frame 816 by releasing the second fastener 822, and the second fastener 822 may be configured to cooperate with a standard camera mount system, such that the portable germicidal lamp 800 may be mounted to a camera tripod, a light stand, loudspeaker stand, or any other structure with a similar mounting system. In this way, a user can position the portable germicidal lamp 800 at any desired height. Additionally, with the base 820 separated from the support frame 816, the housing can be inverted so that the support frame is above the housing, and the support frame adapted to fasten the lamp to an overhead structure, or ceiling.

Finally, according to an embodiment, the entire support structure 414 may be separated from the housing 802 by releasing both first fasteners 818. The housing can then be mounted flush to a horizontal surface, such as a wall, using any of a number of methods and fasteners, including those described with reference to the lighting fixture described above with reference to FIGS. 1-5.

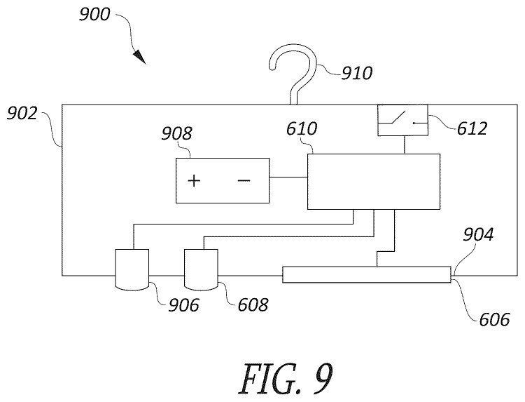

FIG. 9 is a diagrammatic view of a portable germicidal lamp 900, according to another embodiment. The lamp 900 includes a housing 902, with an aperture 904 and an ultraviolet light emitter 606 positioned therein. The light emitter 606 is configured to produce ultraviolet light within a range of between 200 nanometers and 235 nanometers wavelength. First and second sensors 608, 906 are mounted in the housing 802. The first sensor 608 is configured to detect the presence of a human in a defined treatment area, and the second sensor 906 is configured to determine or measure a distance from the second sensor to a target surface. The portable germicidal lamp 900 further includes an electrical controller 610, a switch 612, and an electrical battery 908. The controller 610 is operatively coupled to the light emitter 606, the first and second sensors 608, 906, the switch 612, and the battery 906. A support structure 910 is coupled to the housing 902 and configured to support the portable germicidal lamp 900 in a selected, typically temporary, position. In FIG. 9, the support structure 910 is shown as a hook, such as might be used to hang the portable germicidal lamp 900 from a horizontal bar or rod, etc. However, this is provided as a simple example. Many other support structures and systems have been shown and described elsewhere in the present disclosure as further examples. A person having ordinary skill in the art may devise many further examples that are within the scope of the principles disclosed herein.

In operation, a user may first select an appropriate location to position the portable germicidal lamp 900. Once the lamp 606 is securely positioned, the controller 904 is powered up and a treatment area is defined. This may be done by activating the second sensor 906 and mapping the space in front of the portable lamp 900. Using distances from the device, as determined by the second sensor 906, and angles of deflection from normal to the front face of the light emitter 606, the user may establish the relative intensity of the ultraviolet energy that will impinge upon a surface positioned at any given location throughout the space. With this information, and given the output spectrum and strength of the particular light emitter 606, the user may then define the boundaries of the treatment area, within which a desired level of germicidal action will be obtained. The user may also determine the boundaries within which a degree of caution is required to protect individuals who may pass into the space.

Alternatively or additionally, the controller 610 may be configured to determine the distance to the nearest human, and to modify the output of the lamp 606 to reduce the likelihood of injury.

According to an embodiment, a portable germicidal lamp system is provided, that comprises a housing 902, a UV-C light source 606 disposed in the housing and configured to output ultraviolet light into a space at least intermittently occupied by one or more persons, a power supply configured to provide AC waveform power to the UV-C light source, and an exposed interface configured to receive actuation to couple the power supply to the UV-C light source.

According to an embodiment, a controller is provided, configured to control coupling of the power supply to the UV-C light source and operatively coupled to the exposed interface. The exposed interface may include an on-off switch disposed substantially coincident with an exterior surface of the housing 902. Furthermore, the controller may include a digital communications interface, which may be wholly internal to the housing, or may be part of the exposed interface. The digital communications interface may be configured to receive at least “on” and “off” commands from a remote device.

The portable germicidal lamp 900 may include a support mechanism, as indicated above, configured to temporarily support the germicidal lamp system to illuminate the space 110. The power supply may include a power cord configured to be plugged into an AC socket. Additionally or alternatively, the power supply may include a battery.

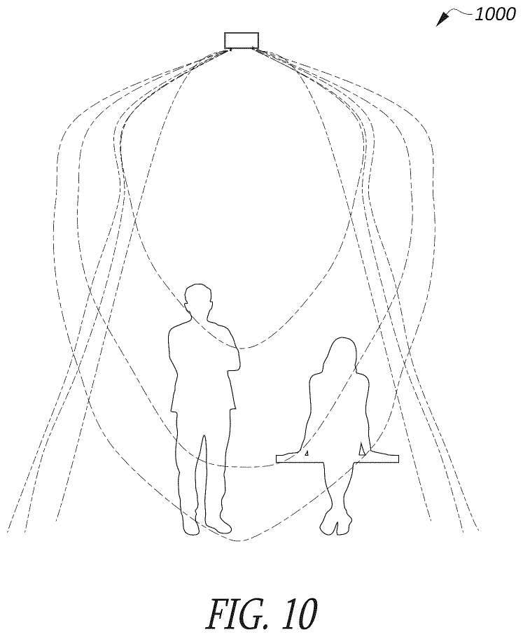

FIG. 10 is a chart 1000 showing levels of UV intensity or strength at various output levels, according to an embodiment. According to one embodiment, a user may use a table providing similar information, but including details such as levels of exposure over time, etc., to determine how to position the device, etc. According to another embodiment, the controller may be configured to regulate the operation of the UV light source to protect individuals who approach the treatment area.

Referring to figures and description above, an auto-ranging UV-C source, according to an embodiment, includes a housing 102, 202, 602, 802, 902; an excimer light source 108, 606, disposed in the housing and configured to output UV-C light to kill pathogens in an exposure space 110, 604, 1000 having a centerline and extending away from the housing 102, 202, 602, 802, 902, and an electronic controller 502, 610 disposed at least partially in the housing 102, 202, 602, 802, 902 and operatively coupled to the excimer light source 108, 606. A distance sensor 808, 906 is operatively coupled to or integrated with the electronic controller 502, 610, the distance sensor 808, 906 being aligned to the centerline of the exposure space 110, 604, 1000 and configured to measure one or more distances from the housing 102, 202, 602, 802, 902 or the excimer light source 108, 608 to a surface bounding the exposure space and/or a person in the exposure space. The electronic controller 502, 610 may be configured to receive data corresponding to the one or more distances from the distance sensor 808, 906 and to control the excimer light source 108, 606 to control a UV-C power intensity according to a pre-determined exposure limit at the person, the surface, or the person and the surface.

Various types of sensors 808, 906 are contemplated. For example, the distance sensor 808, 906 may include an ultrasonic distance sensor, a LiDAR distance sensor, and/or an infrared or visible light distance sensor.

The excimer light source 108, 606 may include an actuated UV-C filter 702 configured to selectively attenuate or occlude UV-C light from the excimer light source 108, 606. The electronic controller 502, 610 may be configured to actuate the UV-C filter 702 to attenuate the UV-C light to meet the exposure limit. Additionally or alternatively, the excimer light source 108, 606 may include selectively energized krypton-halogen emission tubes 606a, 606b. The electronic controller may be configured to selectively energize the krypton-halogen emission tubes to meet the exposure limit.

In the drawings, some elements are designated with a reference number followed by a letter, e.g., “306a, 306b.” In such cases, the letter designation is used where it may be useful in the corresponding description to refer to or differentiate between specific ones of a number of otherwise similar or identical elements. Where the description omits the letter from a reference, and refers to such elements by number only, this can be understood as a general reference to the elements identified by that reference number, unless other distinguishing language is used.

The abstract of the present disclosure is provided as a brief outline of some of the principles of the invention according to one embodiment, but is not intended as a complete or definitive description of any single embodiment thereof, nor should it be relied upon to define terms used in the specification or claims. The abstract does not limit the scope of the claims.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

1-17. (canceled)

18. The germicidal lamp system of claim 19, wherein the light emitter and the electrical controller are positioned within a housing; and further comprising:
the housing including a front housing and a back housing, the front and back housings being configured to contain electrical components of an ultraviolet luminaire; and
a light shield, formed in one or both of the front housing and back housing and configured to substantially prevent heat and ultraviolet light produced by the ultraviolet emitter from reaching the electronic controller.

19. A germicidal lamp system configured to output ultraviolet illumination into a defined treatment area at least intermittently occupied by one or more humans, comprising:
a light emitter configured to output ultraviolet light into a defined treatment area;
a sensor configured to detect the presence of a human in the defined treatment area; and
an electrical controller operatively coupled to the light emitter and the sensor, and configured to control the light emitter responsive to a signal from the sensor indicating the presence of a human in the treatment area.

20-23. (canceled)

24. The lamp system of claim 19, wherein the sensor includes one or more of: an ultrasonic sensor, an infrared sensor, an optical sensor, and an RFID sensor.

25. The lamp system of claim 19, wherein the electrical controller is configured, while a signal from the sensor indicates the presence of a human in the treatment area, to control the light emitter to operate in a safe mode;
wherein operation in the safe mode includes one or more of: operating at a reduced intensity, operating within one or more selected wavelength ranges, and/or limiting a maximum duration of operation in the safe mode over a selected time period.

26. (canceled)

27. The lamp system of claim 19, wherein the sensor is configured to identify specific humans in the treatment area; and
wherein the electrical controller is configured to control the light emitter to output ultraviolet UV-C illumination while the sensor indicates the presence of a human in the treatment area, and to expose no individual human to more than an exposure limit per day of said illumination.

28. (canceled)

29. The lamp system of claim 19, wherein the electrical controller is configured to control the light emitter to output ultraviolet illumination only between 200 nanometers and 235 nanometers wavelength while a signal from the sensor indicates the presence of at least one human in the treatment area.

30. The lamp system of claim 19, wherein the electrical controller is configured to control the light emitter to output ultraviolet illumination only between 200 nanometers and 230 nanometers wavelength while a signal from the sensor indicates the presence of at least one human in the treatment area.

31. The lamp system of claim 19, wherein the electrical controller is configured to control the light emitter to output ultraviolet illumination between 200 nanometers and 280 nanometers wavelength while no human is detected in the treatment area.

32. The lamp system of claim 19, wherein the light emitter includes a first light emitter configured to output ultraviolet illumination between 200 nanometers and 230 or 235 nanometers wavelength and a second light emitter configured to output ultraviolet illumination between nanometers and nanometers wavelength; and
wherein the electrical controller is configured to control the second light emitter to shut down while a signal from the sensor indicates the presence of at least one human in the treatment area.

33. The lamp system of claim 19, further comprising:
a wavelength filter configured to block selected wavelengths of ultraviolet light and further configured to be selectively interposed between the ultraviolet light emitter and the treatment area; and
an actuator operatively coupled to the wavelength filter and to the electrical controller; and
wherein the electrical controller is configured to control the actuator to interpose the wavelength filter between the ultraviolet light emitter and the treatment area while a signal from the sensor indicates the presence of at least one human in the treatment area.

34. The lamp system of claim 33, wherein the wavelength filter is configured to block ultraviolet light between 231 nanometers and 280 nanometers wavelength.

35. The lamp system of claim 33, wherein the wavelength filter is configured to block ultraviolet light between 236 nanometers and 280 nanometers wavelength.

36-39. (canceled)

40. A portable germicidal lamp system, comprising:
a housing;
a UV-C light source disposed in the housing and configured to output ultraviolet light into a space at least intermittently occupied by one or more persons;
power supply configured to provide AC waveform to the UV-C light source; and
an exposed interface configured to receive actuation to couple the power supply to the UV-C light source.

41. The portable germicidal lamp system of claim 40, further comprising:
a controller configured to control coupling of the power supply to the UV-C light source and operatively coupled to the exposed interface.

42. The portable germicidal lamp system of claim 41, wherein the exposed interface includes an on-off switch disposed substantially coincident with an exterior surface of the housing.

43. The portable germicidal lamp system of claim 41, wherein the controller includes a digital communications interface; and
wherein the exposed interface incudes the digital communications interface; and
wherein the communications interface is configured to receive at least “on” and “off” commands from a remote device.

44. (canceled)

45. The portable germicidal lamp system of claim 40, further comprising:
a support mechanism configured to temporarily support the germicidal lamp system to illuminate the space 110.

46. The portable germicidal lamp system of claim 40, wherein the power supply includes a power cord configured to be plugged into an AC socket.

47. The portable germicidal lamp system of claim 40, wherein the power supply includes a battery.

48. An auto-ranging UV-C source, comprising:
a housing;
an excimer light source disposed in the housing and configured to output UV-C light to kill pathogens in an exposure space having a centerline and extending away from the housing;
an electronic controller disposed at least partially in the housing and operatively coupled to the excimer light source; and
a distance sensor operatively coupled to or integrated with the electronic controller, the distance sensor being aligned to the centerline of the exposure space and configured to measure one or more distances from the housing to a surface bounding the exposure space, a person in the exposure space; or to the surface and the person;
wherein the electronic controller is configured to receive data corresponding to the one or more distances from the distance sensor and to control the excimer light source to control a UV-C power intensity according to a pre-determined exposure limit at the person, the surface, or the person and the surface.

49-53. (canceled)
